(19) United States
(12) Patent Application Publication
(10) Pub. No.: US 2023/0356050 A1
(43) Pub. Date: Nov. 9, 2023

(54) A GRIP ANALYSIS SYSTEM AND METHOD

(71) Applicant: EATON INTELLIGENT POWER LIMITED, Dublin, 4 (IE)

(72) Inventors: Daniel ZUCCHETTO, Dublin (IE); Padhraig RYAN, Kilkenny, Keatingstown (IE); Om TANDON, Dublin 1 (IE)

(21) Appl. No.: 18/042,683

(22) PCT Filed: Jan. 15, 2021
(86) PCT No.: PCT/EP2021/050866

§ 371 Date: Feb. 23, 2023

(30) Foreign Application Priority Data
Aug. 28, 2020 (GB) 2013580.2

(51) Int. Cl. A63B 60/46; A63B 53/14

(52) U.S. Cl. CPC A63B 60/46; A63B 53/14; A63B 2060/464

(57) ABSTRACT

One of the most important factors affecting the performance of athletes in club, bat or racket based sports is the athlete's grip on their club, bat or racket. Minor changes in grip position and force can have a significant on the outcome of a shot or other sporting action. Typically, athletes receive feedback on their grip, and the resulting shot, through coaching or practice. However, it is difficult for inexperienced athletes and coaches to correctly diagnose and fix grip faults. The present invention provides a grip analysis system, and method of use thereof, including an array of pressure sensors configured to detect a grip of a user on an object and a processor operable to analyse data from the array of pressure sensors and output at least one grip quality indicator corresponding to the grip of the user on the object.

### FIELD OF THE INVENTION

The present invention relates to a grip analysis system and method and finds particular, although not exclusive, utility in a system and method for providing an athlete, such as a golfer or tennis player, with feedback related to their grip on their sports equipment, such as a golf club or tennis racket.

### BACKGROUND TO THE INVENTION

One of the most important factors affecting the performance of athletes in club, bat or racket based sports is the athlete's grip on their club, bat or racket. Minor changes in grip position and force can have a significant impact on the outcome of a shot or other sporting action. For example, in golf, a shot taken with a minor change in a golfer's grip, such as a 1° change in angle around the shaft, may result in a 10 metre change in position of the ball after the shot. Golfers, along with other athletes, may vary their grip depending on their desired shot outcome. Typically, athletes understand that altering their grip will alter the shape, flight and distance of their shot. Some athletes may aim to use a highly consistent grip placement and force whilst altering some other aspect of their swing. For a right-handed golfer, a swing with a so-called strong grip, which is a term used to describe a grip in which the golfer's left thumb and index finger align with their shoulder and/or neck when addressing a shot, may result in the ball travelling further left than the same swing made with a so-called neutral or weak grip. A strong grip is also understood to close a clubface and effectively reduce the loft of the club, resulting in a shot that flies lower and travels further when compared to a shot made with a neutral or weak grip. Accordingly, the athlete's grip has a large impact on the shot outcome.

Typically, athletes receive feedback on their grip, and the resulting shot, through coaching or practice, often including video feedback. However, the athlete's grip is not the only factor that affects the outcome of their shot. For example, the swing path of a golf shot and environmental factors such as wind also have a significant effect on the outcome of the shot. As there are many factors affecting shot outcomes and small changes in the athlete's grip can have a large impact on the shot outcome, it is difficult for inexperienced athletes and coaches to correctly diagnose and fix grip faults. Furthermore, even elite level athletes and coaches may find it difficult to correctly diagnose and fix grip faults.

Therefore, it is desirable to provide a grip analysis system and method capable of providing feedback related to a user's grip on an object. Objects and aspects of the present invention seek to provide such a system and method.

### SUMMARY OF THE INVENTION

According to a first aspect of the present invention, there is provided a grip analysis system comprising: a sleeve positionable, in use, on an object configured to be gripped by a user; a distributed array of pressure sensors arranged to detect a pressure applied to the sleeve; and a processor operable to: detect, with the array of pressure sensors, a grip of a user on the sleeve; analyse the grip of the user on the sleeve, by: receiving input data from the array of pressure sensors; weighting the input data with a predetermined weight array to determine weighted pressure data; and determining at least one grip quality indicator corresponding to the grip of the user on the sleeve based on the weighted pressure data; and output the at least one grip quality indicator corresponding to the grip of the user on the sleeve.

A key advantage of the present invention is that the system may provide a user with feedback related to their grip on an object. Furthermore, the user may use the feedback provided by the present invention to adjust their grip on the object, such as their hand position and force applied. The feedback may, for example, be visual, audible or tactile.

The object may be a golf club. The sleeve may be a golf club grip. Accordingly the at least one grip quality indicator may relate to a user's golf grip. For example, the at least one grip quality indicator may indicate that the user has a weak, a neutral, or a strong grip. Alternatively, or additionally, the at least one grip quality indicator may indicate that the user is applying too much or too little pressure to the whole or a part of the golf grip.

Alternatively, the object may be another piece of sports equipment, such as a baseball bat, a tennis racket, a badminton racket, a cricket bat, a hockey stick, a hurley, a lacrosse stick, a table tennis paddle, a fishing rod, or any other known sports equipment configured to be held by a user. Accordingly, a user may obtain some feedback related to their grip of the sports equipment.

Alternatively, the object may be a training aid for an automated system, such as a robotic arm or other mechanical device configured to grip an object. The automated system may be programmed to grip the training aid and the grip analysis system may provide feedback related to the grip of the automated system on the training aid. Accordingly, the automated system may be programmed to grip in a desired way. As a further alternative, the object may be a medical device intended for use in detecting a medical problem, such as a neurological or physiological problem. For example, a user may be asked to grip the medical device in a certain way, and the at least one grip quality indicator may be used to determine whether the user's grip is as intended, or if some neurological problem exists. A difference between the user's grip and the intended grip may indicate a neurological or other medical problem.

Alternatively, the object may be a tool. A user may grip the tool to operate it. The sleeve may be a grip on the tool intended to be held by the user. The user may find that a particular grip results in improved operation of the tool. For example, a user may find that they are more likely to drill a hole in a straight line with minimal damage to surrounding materials by using one particular grip, when compared to another grip the user may use. Alternatively, the tool may be a craft tool, such as a craft knife or a tool used in carpentry. A user may find that they are able to achieve more preferable results when using the craft tool with a certain grip, and the system may be used to provide feedback on their grip. Accordingly, the system may be used to allow the user to adjust their grip to the grip which provides the more preferable results.

Weighting the input data may comprise multiplication of the input data by the predetermined weight array. Weighting the input data may comprise multiplication by a further weight array. Weighting the input data may comprise multiplication by one or more weight arrays more than once. Weighting the input data may comprise applying one or more transformations. The transformation(s) may be a rectified linear unit transformation, a sigmoid transformation, a softmax function, or any other known transformation.

The predetermined weight array may be determined via a trained neural network, a random forest algorithm, and/or a gradient boosted decision tree. The predetermined weight array may be determined at least in part via a convolutional neural network. Data may be collected and processed to determine the predetermined weight array.

Preferably, the data used to create the predetermined weight array is stored remotely from the processor. Preferably, the predetermined weight array is stored locally with the processor, although the predetermined weight array may be stored remotely from the processor. The dataset may be large, unwieldly or otherwise better suited to remote storage, meaning it is preferable to store the dataset away from the user's local device. The predetermined weight array and/or data used to create the predetermined weight array may be stored in a cloud-based data storage. In this way, the processor may have access to the most up to date version of the predetermined weight array. The processor may have read-only access to the predetermined weight array. Alternatively, the processor may be configured to edit the predetermined weight array and/or data used to create the predetermined weight array. Alternatively, or additionally, the predetermined weight array and/or data used to create the predetermined weight array may be stored locally to the processor. The predetermined weight array and/or data used to create the predetermined weight array may be stored in a memory coupled to the processor. In this way, the processor may be operable offline, in that no internet or other network connection is used.

The determination of the predetermined weight array may comprise collecting labelled data. The determination of the predetermined weight array may comprise collecting pressure data with the array of pressure sensors corresponding to a grip on the object. The determination of the predetermined weight array may comprise collecting further data with a further pressure sensor. The further pressure sensor may be positioned, in use, on a user's hand. For example, the further pressure sensor may be positioned on, in, or under a glove configured to be worn by a user. Alternatively, or additionally, the determination of the predetermined weight array may comprise a user input. For example, a user, such as an expert athlete or coach, may provide some input related to their grip on the sleeve. The determination of the predetermined weight array may comprise specifying a neural network, random forest algorithm and/or gradient boosted decision tree structure. The determination of the predetermined weight array may comprise training the neural network, random forest algorithm and/or gradient boosted decision tree. The determination of the predetermined weight array may comprise specifying a predetermined accuracy threshold. The determination of the predetermined weight array may comprise comparing the accuracy of the trained neural network, random forest algorithm and/or gradient boosted decision tree with the predetermined accuracy threshold. If the accuracy is found to be acceptable, the model may be stored for use in analysing pressure sensor data. If the accuracy is found to be unacceptable, the model may be respecified and/or retrained. Accordingly, the model may be respecified to define a new model architecture that may be better suited to the task. Furthermore, the model may be retrained with a larger or otherwise improved dataset.

The at least one grip quality indicator may be related to one or more selected from the range: a relative strength or neutrality of hand placement on the grip, a force level, a force position, a maximum force value, a hand angle, a relative angle between two hands, a relative angle between two fingers, a maximum force applied by each hand and a maximum force applied by each finger. Accordingly, the user may be provided with feedback relevant to their grip on the object. Furthermore, the feedback provided may be specific to the sport or other application in which the user participates. The at least one grip quality indicator may be related to a change over time of one or more selected from the range: a relative strength or neutrality of hand placement on the grip, a force level, a force position, a maximum force value, a hand angle, a relative angle between two hands, a relative angle between two fingers, a maximum force applied by each hand and a maximum force applied by each finger. Accordingly, the user may be provided with feedback related to their grip throughout an action. For example, a golfer may be provided with feedback related to their grip throughout their swing, or only a portion of their swing such as the address, backswing, downswing, impact or follow through. The at least one grip quality indicator may be related to an average of data over time of one or more selected from the range: a relative strength or neutrality of hand placement on the grip, a force level, a force position, a maximum force value, a hand angle, a relative angle between two hands, a relative angle between two fingers, a maximum force applied by each hand and a maximum force applied by each finger.

The system may be configured to provide a grip quality indicator for each portion of a user's grip. For example, a grip quality indicator may be provided for each finger and the palm. In this way, the user may be provided with more in-depth feedback that may allow the user to adjust their grip more accurately.

The system may be configured to provide data and/or feedback to a user in real time. Alternatively, or additionally, the system may be configure to provide historical data to a user. In this way, the user may track their progress or recall and recreate previous grips saved in the historical data.

The system may further comprise a feedback device. The feedback device may be configured to receive the at least one grip quality indicator output by the processor. The feedback device may be operable to provide a user gripping the object with feedback related to the at least one grip quality indicator corresponding to the grip of the user on the sleeve. The feedback device may be operably connected to the processor. The feedback device may be physically or wirelessly connected to the processor. The feedback device may be adjacent the sleeve. For example, the feedback device may be on, embedded into, or under the sleeve. Alternatively, or additionally, the feedback device may be separate from the sleeve and distanced from the object. For example, the feedback device may be positionable on the user away from their hands. The feedback device may comprise a first feedback portion adjacent to the sleeve and a second feedback portion separate and spaced from the sleeve.

The feedback device may comprise a haptic feedback device. The haptic feedback device may be operable to provide a user gripping the object with haptic feedback. Haptic feedback may be any form of feedback that the user is able to feel. The haptic feedback device may be on, embedded into, or under the sleeve. As such, the feedback device may provide a user gripping the object with a feeling related to their grip via their hands. The haptic feedback device may be configured to operate by changing temperature, applying force, vibrating, or any other form of mechanical motion, and/or otherwise actuating to provide feedback. The haptic feedback device may be distributed across the sleeve. The haptic feedback device may be distributed across a portion of the sleeve covered by the array of pressure sensors. In this way, the feedback may relate to any portion of the user's grip. Alternatively, the haptic feedback device may be positioned away from the sleeve. For example, the haptic feedback device may be positioned on a wearable object, such as a wristband, an arm band or a glove.

Alternatively or additionally, the feedback device may comprise a visual feedback device operable to provide a user gripping the object with visual feedback. The visual feedback device may comprise a display. The display may be wearable, such as eyewear, or standalone. The visual feedback device may comprise a smart phone or a smart watch. For example, the smart phone or smart watch screen may be used to provide visual feedback.

Alternatively or additionally, the feedback device may comprise an audible feedback device operable to provide a user gripping the object with audible feedback. The audible feedback device may comprise a speaker. The speaker may comprise a loudspeaker, headphones and/or earphones.

The processor may be operable to determine a difference between the determined at least one grip quality indicator and a predetermined grip quality indicator corresponding to a predetermined desired grip. A quality of the feedback may be related to a required grip change to achieve the predetermined desired grip. In this way, the user may be provided with feedback that allows then to adjust their grip to achieve their desired grip. For example, the user may desire a neutral golf grip and the at least one grip quality indicator may indicate that the user currently has a weak grip. Accordingly, the feedback may suggest strengthening the user's grip. As a further example, the user may desire a grip with a moderate pressure and the at least one grip quality indicator may indicate that the user is applying a greater pressure than the desired pressure. Accordingly, the feedback may suggest loosening the user's grip.

The feedback device may be configured to provide a first feedback related to a first grip quality indicator and provide a second feedback related to a second grip quality indicator.

The first and second feedbacks may be provided by a single haptic, visual or audible feedback device. Alternatively, the system may comprise two haptic, visual, audible feedback devices, or a combination thereof, wherein a first feedback device is configured to provide the first feedback and a second feedback device is configured to provide the second feedback.

The processor may be configured to separate the input data into a plurality of input data subsets. The processor may be configured to attribute each input data subset to a portion of a user's hand with multiclass classification. The processor may be configured to identify a position of each user hand portion on the sleeve based on the input data subset attributed to each user hand portion. The processor may be configured to compare the identified positions of each user hand portion to a predetermined desired position of each hand portion in order to identify a difference between the identified positions of each user hand portion and the predetermined desired position of each hand portion. The at least one grip quality indicator may be related to the difference between the identified positions of each user hand portion and the predetermined desired positions of each user hand portion.

The processor may be operatively connected to the array of pressure sensors. Accordingly, the processor may be able to communicate with the array of pressure sensors. The processor may be adjacent to the sleeve. Alternatively, the processor may be spaced and separate from the sleeve. The processor may be an edge computing device.

The predetermined labelled dataset and/or the predetermined weight array may be stored on a remote server. The processor may be in communication with the remote server. The remote server may be in communication with at least one other grip analysis system.

The grip analysis system may further comprise a rechargeable battery configured to supply power to the processor. Alternatively, or additionally, the grip analysis system may comprise a non-rechargeable battery configured to supply power to the processor. Alternative power storage devices, such as supercapacitors, are envisaged.

The array of pressure sensors may comprise at least 8 pressure sensor elements. The array of pressure sensors may be arranged in an 8×1 grid pattern. The array of pressure sensors may comprise at least 368 pressure sensor elements. The array of pressure sensors may be arranged in an 8×46 grid pattern. The array of pressure sensors may comprise at least 1000 sensors. The sensors may be provided at a density of at least 1 sensor per square centimetre, preferably at least 2 sensors per square centimetre, more preferably at least 4 sensors per square centimetre. Each sensor element may have a size of approximately 0.5 centimetres by 0.5 centimetres. Accordingly, providing 4 sensors per square centimetre at a size of 0.5 centimetres by 0.5 centimetres may cover the entire area with sensor elements. Other sensor element sizes and densities are envisaged. The array of pressure sensors may be arranged in a regular grid pattern. Alternatively, the array of pressure sensors may be arranged in an irregular grid pattern. Accordingly, more sensors may be provided in the regions of the sleeve which are more likely to be gripped by the user. For example, if the sleeve is a golf grip, it is likely that the user will grip the sleeve in a middle portion away from the ends of the grip. Therefore, more sensors may be provided in the middle portion of the grip.

The processor may be operable to continually output grip quality indicators corresponding to grips of the user on the sleeve. Continually may mean continuously. The processor may be operable to output grip quality indicators corresponding to grips of the user on the sleeve at predetermined time intervals.

The predetermined weight array may be one of a plurality of predetermined weight arrays. Each of the plurality of predetermined weight arrays may be categorised according to hand size and/or shape. The processor may be configured to determine, based on the input data, a hand size and/or shape categorisation of a hand of a user gripping the sleeve. The processor may be configured to select a predetermined weighted array that corresponds to the same hand size and/or shape categorisation as the determined user hand size and/or shape categorisation.

According to a second aspect of the present invention, there is provided a grip analysis method comprising the steps: detecting, by an array of pressure sensors, a grip of a user on a sleeve; analysing the grip of the user on the sleeve by: receiving input data from the array of pressure sensors; weighting the input data with a predetermined weight array to determine weighted pressure data; and determining at least one grip quality indicator corresponding to the grip of the user on the sleeve based on the weighted pressure data;

and outputting the least one grip quality indicator corresponding to the grip of the user on the sleeve.

The grip analysis method may include each or every step carried out during operation of the processor of the first aspect. Accordingly, each feature of the first aspect may be included in the second aspect of the present invention.

### BRIEF DESCRIPTION OF THE DRAWINGS

FIG. 1 is a schematic view of a grip analysis system;

FIG. 2 is a first flow diagram showing a method of training a neural network to provide a weighted array for use by the grip analysis system shown in FIG. 1; and

FIG. 3 is a second flow diagram showing a method of providing feedback to a user of the grip analysis system shown in FIG. 1.

### DETAILED DESCRIPTION

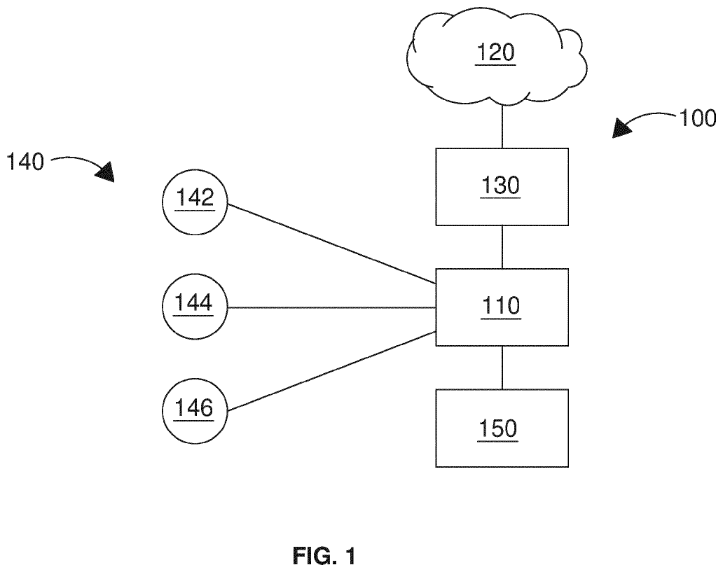

FIG. 1 is a schematic view of a grip analysis system 100. The system includes a processor 110 that is in communication with a cloud-based server 120 via a smart device 130. The processor 110 may be physically or wirelessly connected to the smart device 130 such as a smart phone or a smart watch. For example, the processor 110 and smart device 130 may communicate wirelessly via WiFi or Bluetooth.

The grip analysis system also includes an array of pressure sensors, shown schematically by sensor elements 142, 144, 146. Although only three sensor elements 142, 144, 146 are shown, any number of sensor elements may be provided. For example, 368 sensor elements may be provided in a grid pattern. The array of pressure sensors 140 is configured to be arranged on an object to be gripped by a user, such as a golf club. In this case, the array of pressure sensors may be on, under or embedded in the grip of the golf club or any other connected location. Each sensor element 142, 144, 146 is operable to provide pressure data to the processor 110.

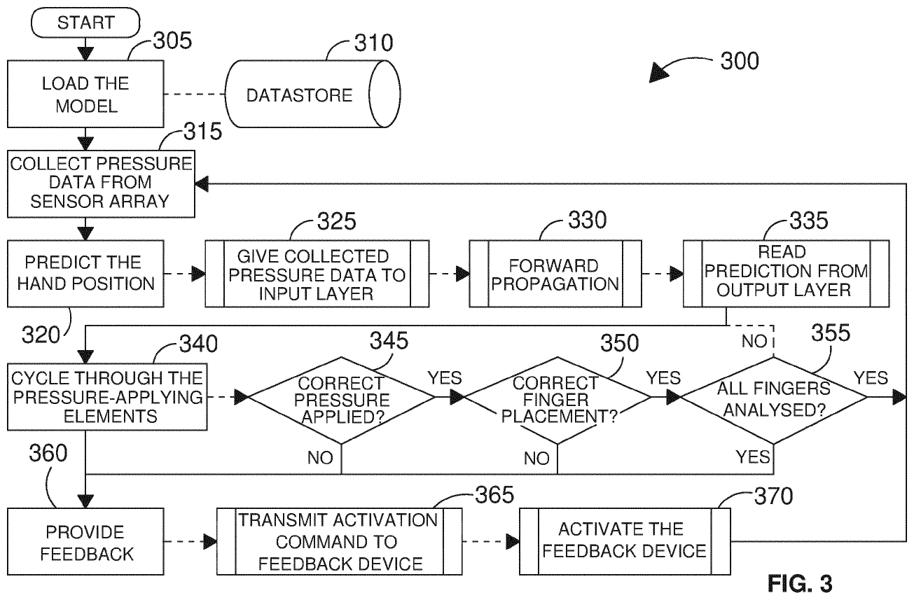

Furthermore, the grip analysis system 100 also includes a haptic feedback device 150. Other types of feedback device 150 are envisaged such as a visual or audible feedback device. The processor 110 is operable to receive pressure data from the array of pressure sensors 140, process the pressure data with a method, to be discussed in more detail with reference to FIG. 3, to obtain a grip quality indicator and operate the feedback device 150 accordingly. The haptic feedback device 150 may be operable to vibrate, heat or cool to indicate to the user that their grip requires adjustment.

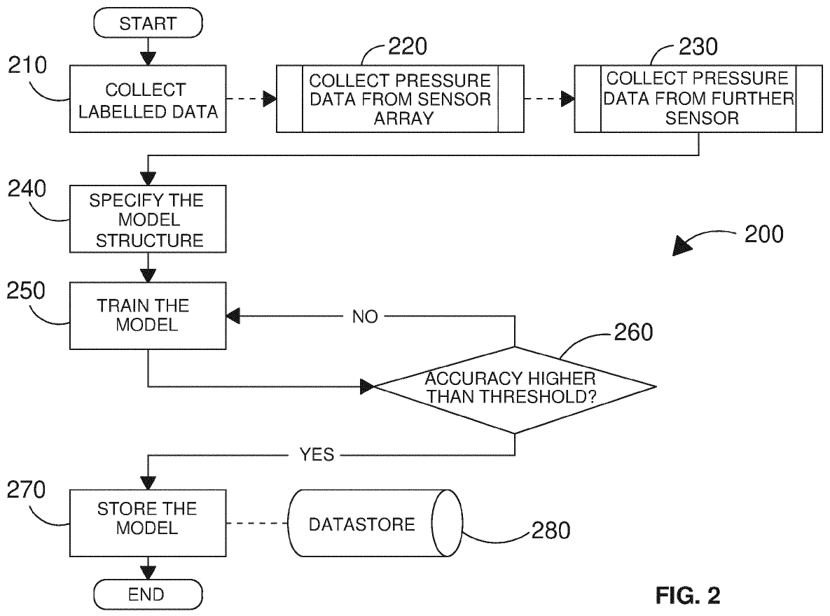

FIG. 2 is a first flow diagram 200 showing a method of training a neural network to provide a weighted array for use by the grip analysis system 100 shown in FIG. 1. The first step of the method 200 is to collect labelled data 210 by having test subjects grip the grip analysis system 100 shown in FIG. 1. The collection of labelled data 210 includes collecting pressure data from the sensor array 220 and collecting pressure data from a further sensor 230. The further sensor may be a glove mounted sensor which is precisely positioned such that a position of the glove, and therefore the user's hand, relative to the array of pressure sensors may be determined. Alternatively, the labelled data 210 may be collected without a further sensor 230. A user, such as an expert athlete or coach, may provide a user input. The user input may be provided during or after the gripping action is performed. For example, a user may review a video recording of a golf swing and classify the grip as strong, neutral or weak.

Once the labelled data has been collected 210, a model structure is specified 240. Specification 240 of the model structure may be an iterative process of trial and error. Space and/or scope may be provided to vary the model architecture and hyperparameters, such as the settings, the learning rate, the regularisation parameter to control overfitting, or any other parameter. The space and/or scope may be user determined and/or determined automatically, such as with automated machine learning type automation. Several models may be specified 240 and trained, then compared to determine relative performance. A relatively better performing model may be chosen. The model structure may be a neural network such as a convolutional neural network. Such a model requires training 250, which is the next step. To train the model 250, the model is tested and an accuracy of the model is compared to a threshold accuracy value 260. If the model does not meet the threshold accuracy value, the model is retrained 250 and retested as described above. The model may be retrained 250 with a larger or otherwise better dataset and/or may be retrained to have a different architecture. Once the accuracy of the model meets the threshold accuracy value, the model is stored 270 in a datastore 280. The datastore is connected to the cloud based server 120 such that the processor 110 of the grip analysis system 100 can access it.

FIG. 3 is a second flow diagram 300 showing a method of providing feedback to a user of the grip analysis system 100 shown in FIG. 1. The method 300 includes loading the model 305 from a datastore 310, such as the model determined and trained by the method 200 of FIG. 2.

The method 300 also includes collecting pressure data from the sensor array 315 when a user is gripping the object to which the sensor array is applied. The pressure data collected may be adjusted or otherwise predicted to reduce or remove sensor noise and/or to take account of degradation over time.

The next step is to predict the user's hand position 320 on the object.

Predicting the hand position 320 includes giving the collected pressure data to the input layer 325 of the trained neural network, forward propagation by the neural network 330, and reading a prediction from the output layer of the neural network 335. Accordingly, a position and force applied by each pressure-applying element, such as each finger, finger portion and/or palm portion, may be predicted or determined.

Once a position and applied force for each pressure-applying element has been determined, a comparison can be made between the determined force and position and a force and position relating to a desired grip. For example, the user may determine that they desire a neutral golf grip, and the method 300 may determine that the user's grip is currently weak. The relative difference between the current and desired grip of the user may be determined.

The next step incudes cycling through the pressure-applying elements 340 to determine which of the pressure-applying elements, such as the user's fingers, are currently positioned incorrectly, or are applying an incorrect force, when compared to the user's desired grip. Cycling through the pressure-applying elements 340 includes determining whether a correct pressure is applied 345, determine whether each finger is correctly placed 350, and determining whether all fingers and hand portions have been analysed 355. Alternatively, the output may be related to a single grip parameter, such as a strong, neutral or weak golf grip, and may be calculated with a single pass with no cycling required. Determining whether the correct pressure is applied 345 and determining whether the fingers are correctly placed 350 may be carried out in any order. The step of determining whether a correct pressure is applied 345 may include determining whether an applied pressure is too high and/or too low.

Each pressure-applying element is considered in turn. If the method 300 determines that the correct pressure is applied 345, the placement of the finger is then considered 350. If the method 300 determines that the finger is correctly placed 350, then a determination is made regarding whether all fingers have been analysed 355. The cycling through the pressure-applying elements 340 continues until a determination is made that all fingers have been analysed 355.

If any pressure-applying element is determined to be providing an incorrect pressure, or is incorrectly positioned, the next step is to provide feedback 360. The provision of feedback 360 includes transmitting an activation command to a feedback device 365, and to activate the feedback device 370 based on the activation command. The feedback device may then provide feedback to the user to adjust their grip such that they may achieve their predetermined desired grip. The method 300 continues to operate, from the step of collecting pressure data from the sensor array 315 to the step of activating the feedback device 370 to continually provide feedback related to the user's grip, which may be adjusted accordingly.

In use, the array of pressure sensors 140 may be arranged on an object. A user may then grip the object in the region covered by the array of pressure sensors 140. Each sensor element 142, 144, 146 may provide a pressure value to the processor 110, which, via processes and methods described herein, is able to determine and output at least one grip quality indicator corresponding to the user's grip on the object. The user may then use the at least one grip quality indicator to adjust their grip. The process may then repeat to provide feedback related to the user's adjusted grip. For example, the array of pressure sensors 140 may be arranged on a golf club grip. A golfer may grip the golf club and address a golf ball. The system 100 may then determine that the golfer is gripping the golf club with a weak grip. However, the golfer may wish to use a neutral grip and adjust their grip accordingly. The system 100 may then reassess the golfer's grip and determine that the golfer is gripping the golf club with a strong grip, having adjusted their grip incorrectly. The golfer may continue to adjust their grip and receive feedback from the system 100 until they are gripping the golf club with their desired grip.

The processor 110 shown in FIG. 1 may be adjacent to the array of pressure sensors 140, or remote from the array of pressure sensors 140. For example, the processor 110 and the array of pressure sensors 140 may both be positioned in the grip of a golf club. Alternatively, the array of pressure sensors 140 may be positioned in the grip of the golf club and the processor 110 may be positioned away from the golf club.

Although the server 120 is described as being cloud-based, it is to be understood that the server 120 may be located alternatively, such as centrally on a private network or locally on a local area network. Furthermore, although a smart phone and a smart watch have been given as examples of a smart device 130, it is to be understood that the smart device 130 may be any device capable of communicating with the processor 110.

The array of pressure sensors 140 may be arranged in a regular grid pattern. Alternatively, the array of pressure sensors 140 may be arranged in an irregular pattern. The array of pressure sensors 140 being configured to be arranged on an object to be gripped by a user may mean that the sensor elements 142, 144, 146 may be in, on or under a portion of the object. Furthermore, although the object has been described as a golf club, it is to be understood that any sporting equipment or other object may be used.

The haptic feedback device 150 is described as being operable to vibrate, heat or cool to indicate to the user that their grip requires adjustment. However, other modes of operation are envisaged. In addition, when alternative feedback devices, such as visual or audible feedback devices, they may be operable to provide visual or audible feedback respectively.

The methods shown in the flow diagrams 200, 300 of FIGS. 2 and 3 are not limited to the steps shown and described above. Additional, or alternative, steps may be undertaken.

Although FIGS. 2 and 3 describe the use of a neural network, other models are envisaged, such as a random forest algorithm or a gradient boosted decision tree. Furthermore, although the further sensor is described as being glove mounted, other positions are envisaged, such as wrist mounted.

Although FIG. 3 includes predicting both the hand position and force applied by the user to the object, only one of these parameters may be predicted and considered. Furthermore, although the pressure-applying elements are described as fingers, finger portions or palm portions, it is to be understood that the pressure-applying elements may be other items, human or non-human, such as a portions of a robotic hand.

In addition, although the feedback device 370 is said to provide feedback continually, it is to be understood that the feedback device 370 may provide feedback only once, a predetermined number of times, or intermittently over a period of time, such as the duration of a swing or hit.

1. A grip analysis system comprising:
a sleeve positionable, in use, on an object configured to be gripped by a user;
a distributed array of pressure sensors arranged to detect a pressure applied to the sleeve; and
a processor operable to:
detect, with the array of pressure sensors, a grip of a user on the sleeve;
analyse the grip of the user on the sleeve, by:
receiving input data from the array of pressure sensors;
weighting the input data with a predetermined weight array to determine weighted pressure data; and
determining at least one grip quality indicator corresponding to the grip of the user on the sleeve based on the weighted pressure data; and
output the at least one grip quality indicator corresponding to the grip of the user on the sleeve.

2. The grip analysis system of claim 1, wherein the object is a golf club and the sleeve is a golf club grip.

3. The grip analysis system of claim 1, wherein the predetermined weight array is determined via a trained neural network, a random forest algorithm, and/or a gradient boosted decision tree.

4. The grip analysis system of claim 3, wherein the predetermined weight array is determined at least in part via a convolutional neural network.

5. The grip analysis system of claim 1, wherein the at least one grip quality indicator is related to one or more selected from the range: a relative strength or neutrality of hand placement on the grip, a force level, a force position, a maximum force value, a hand angle, a relative angle between two hands, a relative angle between two fingers, a maximum force applied by each hand and a maximum force applied by each finger.

6. The grip analysis system of claim 1, further comprising a feedback device configured to receive the at least one grip quality indicator output by the processor, wherein the feedback device is operable to provide a user gripping the object with feedback related to the at least one grip quality indicator corresponding to the grip of the user on the sleeve.

7. The grip analysis system of claim 6, wherein the feedback device comprises a haptic feedback device operable to provide a user gripping the object with haptic feedback.

8. The grip analysis system of claim 6, wherein the feedback device comprises a visual and/or audible feedback device operable to provide a user gripping the object with visual and/or audible feedback.

9. The grip analysis system of claim 6, wherein the processor is operable to determine a difference between the determined at least one grip quality indicator and a predetermined grip quality indicator corresponding to a predetermined desired grip, and wherein a quality of the feedback is related to a required grip change to achieve the predetermined desired grip.

10. The grip analysis system of claim 6, wherein the feedback device is configured to provide a first feedback related to a first grip quality indicator and provide a second feedback related to a second grip quality indicator.

11. The grip analysis system of claim 1, wherein the processor is configured to:
separate the input data into a plurality of input data subsets;
attribute each input data subset to a portion of a user's hand with multiclass classification;
identify a position of each user hand portion on the sleeve based on the input data subset attributed to each user hand portion; and
compare the identified positions of each user hand portion to a predetermined desired position of each hand portion in order to identify a difference between the identified positions of each user hand portion and the predetermined desired position of each hand portion; and
wherein the at least one grip quality indicator is related to the difference between the identified positions of each user hand portion and the predetermined desired positions of each user hand portion.

12. The grip analysis system of claim 1, wherein the processor is operatively connected to the array of pressure sensors and is adjacent to the sleeve.

13. The grip analysis system of claim 12, wherein the predetermined labelled dataset and/or the predetermined weight array is stored on a remote server and the processor is in communication with the remote server.

14. The grip analysis system of claim 13, wherein the remote server is in communication with at least one other grip analysis system.

15. The grip analysis system of claim 12, further comprising a rechargeable battery configured to supply power to the processor.

16. The grip analysis system of claim 1, wherein the array of pressure sensors comprises at least 8 pressure sensor elements.

17. The grip analysis system of claim 16, wherein the array of pressure sensors comprises at least 368 pressure sensor elements.

18. The grip analysis system of claim 1, wherein the processor is operable to continually output grip quality indicators corresponding to grips of the user on the sleeve.

19. The grip analysis system of claim 1, wherein the predetermined weight array is one of a plurality of predetermined weight arrays, wherein each of the plurality of predetermined weight arrays is categorised according to hand size and/or shape, and wherein the processor is configured to:
determine, based on the input data, a hand size and/or shape categorisation of a hand of a user gripping the sleeve; and
select a predetermined weighted array which corresponds to the same hand size and/or shape categorisation as the determined user hand size and/or shape categorisation.

20. A grip analysis method comprising the steps:
detecting, by an array of pressure sensors, a grip of a user on a sleeve;
analysing the grip of the user on the sleeve by:
receiving input data from the array of pressure sensors;
weighting the input data with a predetermined weight array to determine weighted pressure data; and
determining at least one grip quality indicator corresponding to the grip of the user on the sleeve based on the weighted pressure data; and
outputting the least one grip quality indicator corresponding to the grip of the user on the sleeve.
